(19) United States
(12) Patent Application Publication
(10) Pub. No.: US 2003/0134828 A1
(43) Pub. Date: Jul. 17, 2003

(54) Composition and method for increasing in vivo androgen concentration

(76) Inventor: William J. Roberts, Gainesville, FL (US)

Correspondence Address: Stephen T. Sullivan, SULLIVAN LAW GROUP, Suite 1140, 1850 North Central Avenue, Phoenix, AZ, 85004-4586, US

(21) Appl. No.: 10/265,218

(22) Filed: Oct. 3, 2002

Related U.S. Application Data
(63) Continuation-in-part of application No. 10/053,345, filed on Jan. 16, 2002.

(51) Int. Cl. A61K 31/573

(52) U.S. Cl. 514/170

(57) ABSTRACT

A composition is provided including a first prohormone for increasing the concentration of a Class I parent androgen in a subject in vivo, and a second prohormone for increasing the concentration of a Class II parent androgen in the subject in vivo. The first prohormone includes a first carbon-carbon double bond at the 1 position, and a first 17β-hydroxy oxygen appended to the 17 position. The first prohormone further includes a first 17-position promoiety is appended to the first 17β-hydroxy oxygen as a substitute for the 17β-hydroxy hydrogen of the Class I parent androgen. The second prohormone includes a second carbon-carbon double bond at the 4 position and a second 17β-hydroxy oxygen appended to the 17 position. The second prohormone also includes a second 17-position promoiety appended to the second 17β-hydroxy oxygen as a substitute for the 17β-hydroxy hydrogen of the Class II parent androgen.

### RELATED APPLICATION

This application is a continuation-in-part of U.S. patent application Ser. No. 10/053,345, filed in the U.S. Patent & Trademark Office on Jan. 16, 2002.

### BACKGROUND OF THE INVENTION

1. Field of the Invention

The present invention pertains to the field of biochemistry and, more specifically, to the field of stacking or complexing anabolic and/or androgenic steroids and prodrugs or prohormones of anabolic and/or androgenic steroids, and related methods.

2. Description of the Related Art

The importance of having sufficient concentrations of androgenic steroids in the body is well known. The advantages of such steroids can include such things as increased physical performance, improved body composition or increased density, and increased sexual drive or performance, depending upon the specific steroid, organism, etc. It is equally well known that under many common circumstances, deficiencies of these androgenic steroids in the body arise. In humans, for example, as one ages, the normal concentrations of these steroids tend to decrease. Men over the age of about thirty-five typically suffer a reduction in the blood serum concentration of free testosterone. These changes typically result in a reduced general athletic performance and longer time requirements for restoration after extensive exercise, as well as reduced physical and psychological resistance to stress.

A known approach to addressing such steroid concentration deficiencies is to introduce the deficient steroid into the body. In U.S. Pat. No. 5,578,588, for example, methods are disclosed for delivering testosterone supplements, including peroral administration or intramuscular injection of testosterone.

One of the key issues in regard to the usage of anabolic and/or androgenic steroids is the identification of steroid combinations that produce highly beneficial, if not synergistic effects. This issue is discussed in William Roberts, Pharmacological Differences Between Anabolic-Androgenic Steroids (AAS), Anabolic Pharmacology (1999). There it is explained that testosterone administered into the human body becomes bound to androgen receptors. It is observed that there are molecular targets within the human body that are not androgen receptors, yet which function to bind androgen and give a pharmacological response to androgen. These targets may have different binding properties than the androgen receptors. Thus, one anabolic and/or androgenic steroid might be more potent than another at the androgen receptor, but less potent at this other molecular target.

Another issue in regard to the use of anabolic and/or androgenic steroids, such as those described in the above-mentioned U.S. Pat. No. 5,578,588, for example, is that the introduction of certain steroid supplements has been associated with undesirable effects such as poor control over blood concentrations and loss of the steroid due to a “first pass effect,” wherein the steroid is metabolized by the liver prior to reaching general circulation in the blood stream. These losses dramatically reduce the available steroid and, consequently, much higher doses of the steroid supplement generally must be administered to achieve the desired effects. The higher doses sometimes result in an undesirable and unpredictable rise in overall steroid concentration, which, for example, in the case of testosterone, can result in physiological and psychological problems.

Another approach to addressing steroid concentration deficiencies is to introduce a prodrug of the steroid into the body. An example of a prodrug is a prohormone. A prohormone is a compound that itself may have no or substantially no anabolic activity but, when administered in the body, is metabolized or converted into a natural or desired hormone. Such prohormones may become substrates for in vivo bioconversion into the parent compounds, i.e., the corresponding natural or desired hormones. U.S. Pat. No. 5,053,403, for example, discloses that specific prohormones including androstenedione, progesterone, and 17α-β derivatives or analogues can be administered to humans for the purpose of increasing blood concentration of testosterone, reportedly with fewer undesirable effects. Long term use of these prohormones, however, has been also associated with side effects, such as gynecomastia. A pernasal dose of 3.5 mg to 15 mg of these prohormones is reported to increase the blood concentration of testosterone by 34% to 97%. Similarly, U.S. Pat. No. 5,880,117 discloses the use of 4-androstenediol as a peroral testosterone supplement. Androstenedione is a direct precursor of testosterone and estrogen in target tissues having appropriate receptors and enzymes. According to U.S. Pat. No. 6,117,429, androstenediols are precursors for testosterone after oral administration in adults. 19-norandrostenedione is a precursor for 19-nortestosterone, which reportedly has a similar anabolic activity in comparison to testosterone.

The general approach of using prodrugs or prohormones to achieve supplementation of androgenic steroid concentrations in vivo also has been limited, however, in that the effectiveness of such compounds has tended to be relatively low. In some instances, their conversion into the desired steroid is limited, for example, because they are removed from the system through the first pass effect. They also can be converted into undesirable products, for example, as in the case wherein 4-androstenedione is converted into estrogen. Even where the desired bioconversion occurs, the rate of conversion can be sufficiently low that undesirably large quantities of the prodrug must be taken to achieve desired results. The intake of excess amounts of prodrugs can have undesirable side effects.

It is advantageous in many instances to have a prodrug that may be administered in a convenient form, such as by oral, sublingual or pernasal administration. Many prodrugs have not been amenable to such administration, however, for example, because they tend to be broken down prior to absorption in vivo when administered in this fashion.

### OBJECTS OF THE INVENTION

Accordingly, an object of the present invention is to provide compositions and methods that can be used to increase the in vivo concentration and bioavailability of androgens.

Another object of the invention according to certain aspects is to provide compositions and methods that can be used to increase the in vivo concentration and bioavailability of a parent androgen with while being amenable to convenient administration, such as by oral, sublingual or pernasal administration.

Additional objects and advantages of the invention will be set forth in the description that follows, and in part will be apparent from the description, or may be learned by practice of the invention. The objects and advantages of the invention may be realized and obtained by means of the instrumentalities and combinations pointed out in the appended claims.

### SUMMARY OF THE INVENTION

To achieve the foregoing objects, and in accordance with the purposes of the invention as embodied and broadly described in this document, a composition is provided for increasing the concentration of a parent androgen in a subject in vivo in accordance with a first aspect of this invention.

The composition according to this first aspect comprises a first prohormone for increasing the concentration of a Class I parent androgen in a subject in vivo and a second prohormone for increasing the concentration of a Class II parent androgen in a subject in vivo.

The Class I parent androgen comprises a member selected from the group consisting of 5α-androst-1-ene-3α,17β-diol, 5α-androst-1-ene-3β,17β-diol, 5α-estr-1-ene-3α,17β-diol, 5α-estr-1-ene-3β,17β-diol, 17β-hydroxy-5α-androst-1-ene-3-one, and 17β-hydroxy-5α-estr-1-ene-3-one. The Class I parent androgen has a first skeletal structure comprising a 1 position, a 17 position, and a 17β-hydroxy group comprising a 17β-hydroxy oxygen appended to the 17 position and a 17β-hydroxy hydrogen appended to the 17β-hydroxy oxygen. The first prohormone comprises a first substrate having the first skeletal structure of the Class I parent androgen comprising a 1 position and a first 17 position corresponding to the 1 and 17 positions respectively of the Class I parent androgen first skeletal structure. The first substrate comprises a first carbon-carbon double bond at the 1 position and a first 17β-hydroxy oxygen appended to the first 17 position. The first prohormone further comprises a first 17-position promoiety appended to the first 17β-hydroxy oxygen of the substrate as a substitute for the 17β-hydroxy hydrogen of the Class I parent androgen.

The Class II parent androgen comprises a member selected from the group consisting of androst-4-ene-3α,17β-diol, androst-4-ene-3β,17β-diol, estr-4-ene-3α,17β-diol, and estr-4-ene-3β,17β-diol. The Class II parent androgen has a second skeletal structure comprising a 4 position, a 17 position, and a 17β-hydroxy group comprising a 17β-hydroxy oxygen appended to the 17 position and a 17β-hydroxy hydrogen appended to the 17β-hydroxy oxygen. The second prohormone comprises a second substrate having the second skeletal structure of the Class II parent androgen comprising a 4 position and a second 17 position corresponding to the 4 and 17 positions respectively of the Class II parent androgen second skeletal structure. The second substrate comprises a second carbon-carbon double bond at the 4 position and a second 17β-hydroxy oxygen appended to the second 17 position. The second prohormone further comprises a second 17-position promoiety appended to the second 17β-hydroxy oxygen of the substrate as a substitute for the 17β-hydroxy hydrogen of the Class II parent androgen.

According to a second aspect of this invention, a method is provided for increasing the level of parent androgens in a subject, preferably a human being, by administering the composition of the first aspect of the invention described above to the subject.

The method of administration according to the second aspect of the invention may comprise peroral administration, sublingual administration, pernasal administration, or transdermal administration. The method also may comprise complexing the composition with an hydroxypropyl beta cyclodextrin and/or hydroxypropyl gamma cyclodextrin.

Also as part of the method of administration according to this aspect of the invention, the composition preferably is administered once or twice per day, although this is not limiting and other dosage regiments may be preferred depending upon the specific composition, the subject, etc. The composition may be administered in a dosage periodically, preferably for a maximum of two weeks, although longer periods may be practiced. Subsequent to the period of administration, at least two weeks of non-administration preferably follows to permit recovery of natural parent androgen production in the subject.

The composition administration optionally but preferably comprises administering the composition in an amount ranging from 1.0 mg to 1 gram per day. More preferably, the composition administration comprises administering the composition in an amount ranging from 50 mg to 600 mg per day, and even more preferably in an amount ranging from 100 mg to 600 mg per day, and still more preferably 300 mg to 600 mg per day.

In accordance with the first and second aspects of the invention, the first 17-position promoiety and the first 17β-hydroxy oxygen appended to the first 17 position preferably establish a member selected from the group consisting of an alkyloxycarbonyloxy group (of an alkylcarbonate ester), an alkanoyloxy group (of an alkanoate ester), and an alkoxy group (of an alkyl ether), and an alkoxymethyloxy group (of an alkoxy methyl ether). The second substrate, the second 17-position promoiety and the second 17β-hydroxy oxygen appended to the second 17 position preferably establish a member selected from the group consisting of an alkyloxycarbonyloxy group (of an alkylcarbonate ester), an alkanoyloxy group (of an alkanoate ester), an alkyoxy group (of an alkyl ether), and an alkoxymethyloxy group (of an alkoxy methyl ether). The first 17-position promoiety and the second 17-position promoiety may be the same or different from one another. The first and second 17-position promoieties preferably comprise a member selected from the group consisting of a straight-chain alkyl group, branched alkyl group, and cyclic alkyl group, although straight chains are generally, but not necessarily universally, preferred. The first and second 17-position promoieties each preferably have alkyl chain lengths less than 13 carbon atoms, more preferably less than 4 carbon atoms.

In one preferred embodiment of this first and second aspects of the invention, the Class I parent androgen comprises a member selected from the group consisting of 5α-androst-1-ene-3α,17β-diol, 5α-androst-1-ene-3β,17β-diol, 5α-estr-1-ene-3α,17β-diol, and 5α-estr-1-ene-3β,17β-diol, so that a hydroxy group is present at the 3 position of the Class I parent androgen. The first skeletal structure of the Class I parent androgen further comprises a 3 position, and the Class I parent androgen of this preferred embodiment further comprises a 3-hydroxy group comprising a 3-hydroxy oxygen appended to the 3 position and a 3-hydroxy hydrogen appended to the 3-hydroxy oxygen. The first substrate of this preferred embodiment comprises a 3 position corresponding to the 3 position of the Class I parent androgen, and a 3-hydroxy oxygen appended to the 3 position. A 3-position promoiety may be appended to the 3β-hydroxy oxygen of the substrate as a substitute for the 3-hydroxy hydrogen of the Class I parent androgen. The 3-position promoiety and the first 17-position promoiety of the first prohormone may be the same or different from one another, but preferably are the same in this preferred embodiment. Incidentally, compounds identified or referred to herein that identify a bond or functional group at the 3 position, e.g., those identified in this paragraph, may comprise a 3α or a 3β unless indicated otherwise.

In another preferred embodiment of the first and second aspects of the invention, the Class II parent androgen further comprises a 3 position, and the Class II parent androgen further has a 3-hydroxy group comprising a 3-hydroxy oxygen appended to the 3 position and a 3-hydroxy hydrogen appended to the 3-hydroxy oxygen. The second substrate of this preferred embodiment comprises a 3 position corresponding to the 3 position of the Class II parent androgen, and a 3-hydroxy oxygen appended to the 3 position. The second prohormone of this preferred embodiment further comprises a 3-position promoiety appended to the 3β-hydroxy oxygen of the substrate as a substitute for the 3-hydroxy hydrogen of the Class II parent androgen. The 3-position promoiety and the second 17-position promoiety of the second prohormone may be the same or different from one another, but are preferably the same. Incidentally, compounds identified or referred to herein that identify a bond or functional group at the 3 position, e.g., those identified in this paragraph, may comprise a 3α or a 3β unless indicated otherwise.

In yet another preferred embodiment of the first and second aspects of the invention, the first and second prohormones comprise first and second 3-position promoieties, respectively. Alternatively, i.e., instead of having promoieties at the 3 position, the first prohormone may have a hydroxy or ketone moiety appended to the first 3-position carbon, and the second prohormone may have a hydroxy moiety appended to the second 3-position carbon.

In some forms, particularly those intended for peroral administration, it is preferable albeit optional for the composition of the first and second aspects of this invention to include a carrier. The carrier may be a solid, a liquid, a semi-solid, or other suitable form.

In accordance with a third aspect of this invention, a composition is provided, the composition comprising:

(a) a first compound having a first skeletal structure as follows

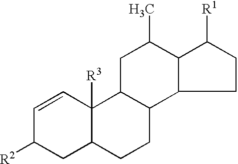

wherein R¹ and R² are the same or different from one another and selected from the group consisting of hydroxyl, —OC(O)OR⁴, —OC(O)R⁴, —OR⁴, —OCH₂OR, and ═O; wherein R³ is selected from the group consisting of hydrogen and methyl; and wherein R⁴ is an alkyl group having less than 13 carbon atoms; and

(b) a second compound having a second skeletal structure as follows

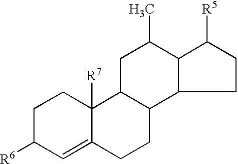

wherein R⁵ and R⁶ are the same or different from one another and selected from the group consisting of hydroxyl, —OC(O)OR⁸, —OC(O)R⁸, —OR⁸, and —OCH₂OR⁸, except that R⁵ and R⁶ are not both hydroxyl; wherein R⁷ is selected from the group consisting of hydrogen and methyl; and wherein R⁸ is an alkyl group having less than 13 carbon atoms.

According to a fourth aspect of this invention, a method is provided for increasing androgen level in a living subject, preferably a human being, by administering the composition of the third aspect of the invention described above.

In accordance with this fourth aspect, the composition administration may comprise peroral administration, pernasal administration, transdermal administration, sublingual administration, and other means. Peroral administration is presently preferred.

As part of the method of this fourth aspect, the composition administration may comprise complexing the compound with an hydroxypropyl beta cyclodextrin, and/or with an hydroxypropyl gamma cyclodextrin.

The composition administration method of the second and fourth aspects of the invention optionally may further comprise the step of applying an enteric coating to the composition prior to administering the composition.

The optional and preferred administration regiments and dosages as noted above in connection with the second aspect apply to the method according to the fourth aspect of the invention as well.

In accordance with the third and fourth aspects of the invention described above, a member selected from the group consisting of R¹ and R² may consists of a ketone, ═O. In the event that R² consists of ═O, that is, a ketone, R¹ preferably consists of a member selected from the group consisting of hydroxyl, —OC(O)OR⁴, —OC(O)R⁴, —OR⁴, and —OCH₂OR⁴, more preferably R¹ consists of a member selected from the group of —OC(O)OR⁴, —OC(O)R⁴, —OR⁴, and —OCH₂OR⁴, and R⁴ preferably consists of a straight-chain alkyl group.

In accordance with another preferred embodiment of the third and fourth aspects of invention, R¹ and R² each consists of -OC(O)OR⁴, with R⁴ preferably consisting of a straight-chain alkyl group. In accordance with yet another preferred embodiment of the third and fourth aspect of the invention, a member selected from the group consisting of R¹ and R² consists of the hydroxyl, more preferably R¹ consists of —OC(O)OR⁴ and R² consists of the hydroxyl, with R⁴ preferably consisting of a straight-chain alkyl group.

In accordance with a further preferred embodiment of the third and fourth aspects of invention, R⁵ and R⁶ each consists of —OC(O)OR⁸, with R⁸ preferably consisting of a straight-chain alkyl group. In accordance with yet another preferred embodiment of the third and fourth aspect of the invention, a member selected from the group consisting of R⁵ and R⁶ consists of the hydroxyl, more preferably R⁵ consists of —OC(O)OR⁸ and R⁶ consists of the hydroxyl, with R⁸ preferably consisting of a straight-chain alkyl group.

In accordance with a fifth aspect of the invention, a composition is provided comprising a first prohormone for increasing the concentration of a Class I parent androgen in a subject in vivo, and pregnenolone. In accordance with a sixth aspect of the invention a method is provided for administering the composition to a subject, such as a human. The Class I parent androgen comprises a member selected from the group consisting of 5α-androst-1-ene-3α,17β-diol, 5α-androst-1-ene-3β,17β-diol, 5α-estr-1-ene-3α,17β-diol, 5α-estr-1-ene-3β,17β-diol, 17β-hydroxy-5α-androst-1-ene-3-one, and 17β-hydroxy-5α-estr-1-ene-3-one. The Class I parent androgen has a first skeletal structure comprising a 1 position, a 17 position, and a 17β-hydroxy group comprising a 17β-hydroxy oxygen appended to the 17 position and a 17β-hydroxy hydrogen appended to the 17β-hydroxy oxygen. The first prohormone comprises a first substrate having the first skeletal structure of the Class I parent androgen comprising a 1 position and a first 17 position corresponding to the 1 and 17 positions respectively of the Class I parent androgen skeletal structure. The first substrate comprises a first carbon-carbon double bond at the 1 position and a first 17β-hydroxy oxygen appended to the first 17 position. The first prohormone further comprises a first 17-position promoiety appended to the first 17β-hydroxy oxygen of the substrate as a substitute for the 17β-hydroxy hydrogen of the Class I parent androgen. The first promoiety of the fifth and sixth aspects of the invention may be any of the first promoieties described above with regard to any of the first, second, third, and fourth aspects.

According to a preferred modification to the fifth and sixth aspects of the invention, the composition further comprises a second prohormone for increasing the concentration of a Class II parent androgen in a subject in vivo. The Class II parent androgen comprises a member selected from the group consisting of androst-4-ene-3α,17β-diol, androst-4-ene-3β,17β-diol, estr-4-ene-3α,17β-diol, and estr-4-ene-3β,17β-diol. The Class II parent androgen has a second skeletal structure comprising a 4 position and a 17 position, and a 17β-hydroxy group comprising a 17β-hydroxy oxygen appended to the 17 position and a 17β-hydroxy hydrogen appended to the 17β-hydroxy oxygen. The second prohormone comprises a second substrate having the second skeletal structure of the Class II parent androgen comprising a 4 position and a second 17 position corresponding to the 4 and 17 positions respectively of the Class II parent androgen skeletal structure. The second substrate comprises a second carbon-carbon double bond at the 4 position and a second 17β-hydroxy oxygen appended to the second 17 position. The second prohormone of the fifth and sixth aspects of the invention further comprises a second 17-position promoiety appended to the second 17β-hydroxy oxygen of the substrate as a substitute for the 17β-hydroxy hydrogen of the Class II parent androgen. The second promoiety of the fifth and sixth aspects of the invention may be any of the second promoieties described above with regard to any of the first, second, third, and fourth aspects.

### DETAILED DESCRIPTION OF THE PREFERRED EMBODIMENTS AND PREFERRED METHODS

Reference will now be made in detail to the presently preferred embodiments and preferred methods of the invention. It should be noted, however, that the invention in its broader aspects is not limited to the specific details, representative compositions and methods, and illustrative examples described in this section in connection with the preferred embodiments and methods. The invention according to its various aspects is particularly pointed out and distinctly claimed in the attached claims read in view of this specification, and appropriate equivalents.

It is to be noted that, as used in the specification and the appended claims, the singular forms “a,” “an,” and “the” include plural referents unless the context clearly dictates otherwise.

In accordance with the first aspect of the invention, a composition is provided for increasing the concentration of parent androgens in a subject in vivo. As noted above, the composition comprises a first prohormone for increasing the concentration of a Class I parent androgen in a subject in vivo. The compound similarly may comprise a plurality of first prohoromones, in which case these prohormones preferably would include a plurality of one or more of the first prohormones as described herein.

The Class I parent androgen comprises a member selected from the group consisting of 5α-androst-1-ene-3α,17β-diol, 5α-androst-1-ene-3β,17β-diol, 5α-estr-1-ene-3α,17β-diol, 5α-estr-1-ene-3β,17β-diol, 17β-hydroxy-5α-androst-1-ene-3-one, and 17β-hydroxy-5α-estr-1-ene-3-one (including mixtures thereof as noted above).

The Class I parent androgen has a first skeletal structure comprising a 1 position, a 17 position, and a 17β-hydroxy group comprising a 17β-hydroxy oxygen appended to the 17 position and a 17β-hydroxy hydrogen appended to the 17β-hydroxy oxygen.

The composition further comprises a second prohormone for increasing the concentration of a Class II parent androgen in the subject in vivo. The compound similarly may comprise a plurality of second prohoromones, in which case there preferably would be a plurality of one or more of the second prohormones as described herein.

The Class II parent androgen comprises a member selected from the group consisting of androst-4-ene-3α,17β-diol, androst-4-ene-3β,17β-diol, estr-4-ene-3α,17β-diol, and estr-4-ene-3β,17β-diol. The composition also may and generally will comprise a plurality of such second parent androgens, in which case mixtures of these Class II parent androgens would be involved.

The Class II parent androgen has a second skeletal structure comprising a 4 position, a 17 position, and a 17β-hydroxy group comprising a 17β-hydroxy oxygen appended to the 17 position and a 17β-hydroxy hydrogen appended to the 17β-hydroxy oxygen. The numbering corresponds to the ring identification and carbon numbering system well known in the field of steroid chemistry, for example, as adopted in the 1989 recommendations of the International Union of Pure and Applied Chemistry (“IUPAC”).

Without wishing to be bound by any particular theory, Class I parent androgens are believed to be generally more reactive with androgen receptors in human beings, as described in William Roberts, Pharmacological Differences Between Anabolic-Androgenic Steroids (AAS) (1999). It is to be noted that, as used in the specification and the appended claims, the term “Class I” shall mean androgenic compounds having a carbon-carbon double bond at the 1 position unless the context clearly dictates otherwise. Also, as used in the specification and the appended claims, the term “Class II” shall mean androgenic compounds having a carbon-carbon bond at the 4 position, but not at the 1 position, unless the context clearly dictates otherwise.

The first prohormone comprises a first substrate having the first skeletal structure of the Class I parent androgen. It thus comprises a 1 position and a first 17 position corresponding to the 1 and 17 positions, respectively, of the Class I parent androgen first skeletal structure. The first substrate comprises a first carbon-carbon double bond at the 1 position and a first 17β-hydroxy oxygen appended to the first 17 position. The first prohormone further comprises a first 17-position promoiety appended to the first 17β-hydroxy oxygen of the first substrate as a substitute for the 17β-hydroxy hydrogen of the Class I parent androgen.

The parent androgen first skeletal structure also preferably comprises a 3 position, and the first substrate similarly also comprises a 3 position corresponding to that of the parent androgen first skeletal structure. Preferably a member selected from the group consisting of a double-bonded oxygen and a first 3-hydroxy oxygen is appended to the 3 position. In the event that the 3 position has the first 3-hydroxy oxygen appended to it, preferably appended to the first 3-hydroxy oxygen is a member selected from the group consisting of a first 3-hydroxy hydrogen and a first 3-position promoiety. As noted, in each instance in this document wherein a group is bonded at the 3 position (at the first or “A” carbon ring) of the first or second prohormone, the 3 position configuration may comprise or consist essentially of either the α configuration or the β configuration, or both.

The first 17-position promoiety and the first 17β-hydroxy oxygen appended to the first 17 position preferably establish an alkyloxycarbonyloxy group (of an alkylcarbonate ester), in accordance with a preferred embodiment of this first aspect. Preferably, the first 17-position promoiety of the alkylcarbonate ester has an alkyl chain with less than 13 carbon atoms, and more preferably less than four carbon atoms. The alkyloxycarbonyloxy group preferably comprises a straight chain or branched alkyl group, and is free of cyclic groups. The alkylcarbonate preferably is selected from the group consisting of methyl carbonate, ethyl carbonate, propyl carbonate, isopropyl carbonate, butyl carbonate, isobutyl carbonate, t-butyl carbonate, valeryl carbonate, hexyl carbonate, heptyl carbonate, octyl carbonate, nonyl carbonate, decyl carbonate, undecyl carbonate, and dodecyl carbonate. The alkylcarbonate may include a cyclic alkyl group such as cyclopentyl methyl carbonate, cyclopentylpropyl carbonate, cyclohexyl methyl carbonate, and cyclohexylpropyl carbonate. Alkyl carbonates having lower carbon chain lengths generally are preferred, although not necessarily universally so. Ethylcarbonate esters generally are even more preferred.

Examples of first prohormones comprising an alkyloxycarbonyloxy group at the 17 position include 5α-androst-1-ene-3-one-17β-alkylcarbonate (e.g., 5α-androst-1-ene-3-one-17β-ethylcarbonate, also known as 17-hydroxy-5α-androst-1-ene-3-one-17 β-ethylcarbonate), 5α-androst-1-ene-3-ol-17β-alkylcarbonate (e.g., 5α-androst-1-ene-3-ol-17β-ethylcarbonate, also known as 5α-androst-1-ene-3,17-diol 17β-ethylcarbonate), 5α-androst-1-ene-3,17 β-di(alkylcarbonate), (e.g., 5α-androst-1-ene-3,17β-di(ethylcarbonate), also known as 5α-androst-1-ene-3,17-diol 3,17β-di(ethylcarbonate)), 5α-estr-1-ene-3-one-17β-alkylcarbonate (e.g., 5α-estr-1-ene-3-one-17β-ethylcarbonate), 5α-estr-1-ene-3-ol-17β-alkylcarbonate (e.g., 5α-estr-1-ene-3-ol-17β-ethylcarbonate), and 5α-estr-1-ene-3,17β-di(alkylcarbonate), (e.g., 5α-estr-1-ene-3,17β-di(ethylcarbonate)).

The synthesis of 5α-androst-1-ene-3,17β-di(alkylcarbonate) may be accomplished by reacting 5α-androst-1-ene-3,17β-diol with 2.00 equivalents each of alkyl chloroformate. Generally, this reaction may be conducted with a base (e.g., 2,6-lutidine, pyridine, and triethylamine) and a solvent (e.g., dichloromethane, acetone, acetonitrile, pyridine, and lutidine). Examples of this synthesis route are provided below. The synthesis of 5α-androst-1-ene-3-one-17β-alkylcarbonate and 5α-androst-1-ene-3-ol-17β-alkylcarbonate may be performed in much the same manner, except that the starting parent androgen may be 5α-androst-1-ene-17β-ol-3-one, which may be reacted with 1.00 equivalent of alkyl chloroformate. The reaction produces 5α-androst-1-ene-3-one-17β-alkylcarbonate, which optionally then may be reduced, for example, with sodium borohydride or the like, to synthesize 5α-androst-1-ene-3-ol-17β-alkylcarbonate. The equivalent estranes may be synthesized in a similar manner, but with a 19-nor-androgen parent compound as a reagent. Androgens such as 5α-androst-1-ene-3,17-diol and 5α-androst-1-ene-17-ol-3-one are commercially available from sources such as LPJ Research, Inc. of Seymour, Ill., and ChemPhar International Inc. of Hillsborough, N.J., among others. 5α-estr-1-ene-17-ol-3-one may be made according to the method of Liston and Howarth (Canadian Journal of Chemistry, 1967, volume 45, pages 2577-2582) also as reported by Ghaffari and Abul-Hajj (Journal of Steroid Biochemistry and Molecular Biology, 1990, volume 37(2), pages 237-44).

The first 17-position promoiety and the first 17β-hydroxy oxygen appended to the first 17 position also may establish an alkanoyloxy group of an alkanoate in accordance with another embodiment of this aspect. The 17-position promoiety of this embodiment preferably is an acyl group having less than 13 carbon atoms, more preferably less than 4 carbon atoms. The 17-position promoiety of the alkanoyloxy group preferably comprises a member selected from the group consisting of a straight-chain acyl promoiety, a branched acyl promoiety, and a cyclic acyl promoiety appended to the first 17β hydroxy oxygen. Exemplary first prohormones including at least one alkanolyloxy group include 5α-androst-1-ene-3-one-17β-alkanoate (e.g., 5α-androst-1-ene-3-one-17β-acetate (also referred to as 17β-acetoxy-5α-androst-1-ene-3-one) and 5α-androst-1-ene-3-one-17β-propionate), 5α-androst-1-ene-3β-hydroxy-17β alkanoate (e.g., 5α-androst-1-ene-3β-hydroxy-17β-acetate and 5α-androst-1-ene-3β-hydroxy-17β-propionate), 5α-androst-1-ene-3,17β-di(alkanoate) (e.g., 5α-androst-1-ene-3,17β-di(acetate) and 5α-androst-1-ene-3,17β-di(propionate)), 5α-estr-1-ene-3-one-17β-alkanoate (e.g., 5α-estr-1-ene-3-one-17β-acetate and 5α-estr-1-ene-3-one-17β-propionate), 5α-estr-1-ene-3β-hydroxy-171β-alkanoate (e.g., 5α-estr-1-ene-3 β-hydroxy-17β-acetate and 5α-estr-1-ene-3β-hydroxy-17β-propionate), and 5α-estr-1-ene-3,17β-di(alkanoate) (e.g., 5α-estr-1-ene-3,17β-di(acetate) and 5α-estr-1-ene-3,17β-di(propionate)).

An acid anhydride reaction or other known ester synthesis routes may be used to prepare these compounds. For example, acetic anhydride may be reacted with the mono-ol or diol parent androgen to prepare, for example, an acetate (ethanoate) of 5α-androst-1-ene-17β-ol-3-one or 5α-androst-1-ene-3,17β-diol, respectively. An excess molar ratio of acid anhydride to parent androgen, for example of about 10:1, preferably is used. Other known ester synthesis routes include the Fischer esterification reaction, in which esters are synthesized by reaction between a carboxylic acid and an alcohol, preferably in an alcohol solution. In each instance in which the first prohormone includes a hydroxy group at the 3 position, the bond at the 3 carbon may be 3α or 3β and preferably comprises a mixture thereof. A separation technique, such as chromotography, is preferably used for separating the 3α and 3β stereoisomers from one another, unless a mixture is desired, as may particularly be the case with prohormone deriviatives of androst-4-ene-3,17β-diol.

According to another embodiment of this aspect of the invention, the first 17-position promoiety and the first 17β-hydroxy oxygen appended to the first 17 position establish an alkoxy group. The alkoxy group has an alkyl group appended to the first 17β-hydroxy oxygen, with the alkyl group preferably having less than 13 carbon atoms, and preferably less than four carbon atoms. For example, the alkoxy group may be selected from methoxy, ethoxy, butoxy, isopropoxy, isobutoxy, t-butoxy, valeroxy, hexanoxy, heptanoxy, octanoxy, nonanoxy, decanoxy, undecanoxy, cyclopentoxy, and cyclopentylpropoxy. The first 17-position promoiety may comprise a member selected from the group consisting of a straight-chain alkyl promoiety, a branched alkyl promoiety, and a cyclic alkyl promoiety appended to the first 17β hydroxy oxygen. The first prohormone, thus, may comprise, for example, 17β-alkoxy-5α-androst-1-ene-3-one, 17β-alkoxy-3β-hydroxy-5α-androst-1-ene, 3,17β-di(alkoxy)-5α-androst-1-ene, 17β-alkoxy-5α-estr-1-ene-3-one, 17β-alkoxy-3β-hydroxy-5α-estr-1-ene, and 3,17β-di(alkoxy)-5α-estr-1-ene.

Alkoxy groups may be prepared by known synthesis techniques, including, not necessarily by limitation, reacting the steroid with a corresponding alkyl iodide in, for example, acetonitrile or dichloromethane.

According to another embodiment of this aspect of the invention, the first 17-position promoiety and the first 17β-hydroxy oxygen appended to the first 17 position establish an alkoxymethyloxy group (of an alkoxy methyl ether), with the alkoxymethyloxy group having an alkyl moiety having less than 13 carbon atoms. More preferably, the alkoxy moiety consists of methoxy. Other representative alkoxy moieties include moieties selected from the group consisting of ethoxy, butoxy, isopropoxy, isobutoxy, t-butoxy, valeroxy, hexanoxy, heptanoxy, octanoxy, nonanoxy, decanoxy, undecanoxy, cyclopentoxy, and cyclopentylpropoxy. Examples of the first prohormone thus would include 17β-methoxymethyl-5α-androst-1-ene-3-ol, 17β-methoxymethyl-5α-androst-1-ene-3-one, 17β-methoxymethyl-5α-estr-1-ene-3-ol, 17β-methoxymethyl-5α-estr-1-ene-3-one, 3,17β-di(methoxymethyl)-5α-androst-1-ene, and mixtures or combinations thereof. In each instance in which the compound includes a hydroxy group at the 3 position, the bond at the 3 carbon may be 3α or 3β and preferably comprises a mixture thereof.

Methoxymethyl ethers of these types may be synthesized using known synthesis techniques, for example, by reaction with iodomethyl methyl ether, or chloromethyl methyl ether, in a Williamson ether synthesis. The compounds may be formed by reaction of the sodium or potassium salt of the parent androgen with chloromethyl methyl ether, bromomethyl methyl ether, or iodomethyl methyl ether in dichloromethane or other suitable solvent.

Without wishing to be bound by any particular theory, it is believed that the Class I androgen may function in some instances as a depressant. To counteract this possible side-effect, the composition may further comprise pregnenolone. When used as an antidepressant, pregnenolone may be present in an amount of, for example and not necessarily limitation, 10 mg to 20 mg per 100 mg of primary prohormone. Pregnenolone may be used with the primary (first) prohormone alone, or in combination with both the primary (first) and secondary (second) prohormones.

The second prohormone comprises a second substrate having the second skeletal structure of the Class II parent androgen comprising a 4 position and a second 17 position corresponding to the 4 and 17 positions respectively of the Class II parent androgen second skeletal structure. The second substrate comprises a second carbon-carbon double bond at the 4 position and a second 17β-hydroxy oxygen appended to the second 17 position. The second prohormone further comprises a second 17-position promoiety appended to the second 17β-hydroxy oxygen of the second substrate as a substitute for the 17β-hydroxy hydrogen of the Class II parent androgen. The second substrate also comprises a 3 position corresponding to a 3 position of the Class II parent androgen second skeletal structure, and preferably a second 3-hydroxy oxygen appended to the 3 position. Preferably appended to the second 3-hydroxy oxygen is a member selected from the group consisting of a second 3-hydroxy hydrogen and a second 3-position promoiety. As noted, in each instance in this document wherein a group is bonded at the 3 position (at the first or “A” carbon ring) of the first or second prohormone, the 3 position configuration may comprise or consist essentially of either the α configuration or the β configuration, or both.

The second 17-position promoiety and the second 17β-hydroxy oxygen appended to the second 17 position preferably establish an alkyloxycarbonyloxy group of an alkylcarbonate ester. Preferably, the second 17-position promoiety of the alkylcarbonate ester has an alkyl group with less than 13 carbon atoms, and more preferably less than 4 carbon atoms. The alkylcarbonate ester preferably comprises a straight chain or branched (non-cyclic) alkyl group, and more preferably is selected from the group consisting of methyl carbonate, ethyl carbonate, propyl carbonate, isopropyl carbonate, butyl carbonate, isobutyl carbonate, t-butyl carbonate, valeryl carbonate, hexyl carbonate, heptyl carbonate, octyl carbonate, nonyl carbonate, decyl carbonate, undecyl carbonate, and dodecyl carbonate. The alkylcarbonate may include a cyclic group, such as cyclopentyl methyl carbonate, cyclopentylpropyl carbonate, cyclohexyl methyl carbonate, and cyclohexylpropyl carbonate. Alkyl carbonate esters having lower carbon chain lengths generally are preferred, although not necessarily universally so. Ethylcarbonate esters generally are even more preferred.

Examples of second prohormones comprising alkylcarbonate ester(s) include androst-4-ene-3-ol-17 β-alkylcarbonate (e.g., androst-4-ene-3-ol-17β-ethylcarbonate, also known as 17β-hydroxyandrost-4-ene-3-ol 17β-ethylcarbonate), androst-4-ene-3,17β-di(alkylcarbonate), (e.g., 5α-androst-4-ene-3,17β-di(ethylcarbonate), also known as androst-4-ene-3,17β-diol-3,17β-di(ethylcarbonate)), estr-4-ene-3-ol-17β-alkylcarbonate (e.g., estr-4-ene-3-ol-17β-ethylcarbonate), estr-4-ene-3,17β-di(alkylcarbonate), (e.g., estr-4-ene-3,17β-di(ethylcarbonate)).

The synthesis of androst-4-ene-3,17β-di(alkylcarbonate) may be accomplished by reacting androst-4-ene-3,17β-diol with 2.00 equivalents each of alkyl chloroformate. Generally, this reaction may be conducted with a base (e.g., 2,6-lutidine, pyridine, or triethylamine) and a solvent (e.g., dichloromethane, acetone, acetonitrile, pyridine, and lutidine). Examples of this synthesis route are provided below. The synthesis of androst-4-ene-3-ol-17β-alkylcarbonate may be performed in much the same manner as 5α-androst-1-ene-3-ol-17β-alkylcarbonate, except that the starting parent androgen is androst-4-ene-17β-ol-3-one, which is commercially available and reacted with 1.00 equivalent of alkyl chloroformate. The reaction produces androst-4-ene-3-one-17β-alkylcarbonate, which optionally may then be reduced, for example, with sodium borohydride or the like, to synthesize androst-4-ene-3-ol-17β-alkylcarbonate. The equivalent estranes may be synthesized in the same manner, but with a 19-nor-androgen parent compound, which are also commercially available.

The second 17-position promoiety and the second 17β-hydroxy oxygen appended to the second 17 position may also establish an alkanoyloxy group of an alkanoate. The 17-position promoiety forming the alkanoyloxy group preferably is an acyl group having less than 13 carbon atoms, more preferably less than 4 carbon atoms. The 17-position promoiety of the alkanoyloxy group preferably comprises a member selected from the group consisting of a straight-chain acyl promoiety, a branched acyl promoiety, and a cyclic acyl promoiety. Exemplary second prohormones including at least one alkanoyloxy group include androst-4-ene-3β-hydroxy-17β-alkanoate (e.g., androst-4-ene-3β-hydroxy-17β-acetate and androst-4-ene-3β-hydroxy-17β-propionate), androst-4-ene-3,17β-di(alkanoate) (e.g., androst-4-ene-3,17β-di(acetate) and androst-4-ene-3,17β-di(propionate)), estr-4-ene-3β-hydroxy-17β-alkanoate (e.g., estr-4-ene-3β-hydroxy-17β-acetate and estr-4-ene-3β-hydroxy-17β-propionate), and estr-4-ene-3,17β-di(alkanoate) (e.g., estr-4-ene-3,17β-di(acetate) and estr-4-ene-3,17β-di(propionate)).

An acid anhydride reaction or other known ester synthesis routes and separation techniques described above in connection with the first prohormones may be used to prepare these second prohormones.

According to another embodiment of this aspect of the invention, the second 17-position promoiety and the second 17β-hydroxy oxygen appended to the second 17 position establish an alkoxy group (of an alkyl ether). The alkoxy group has an alkyl group (as the second 17-position promoiety) appended to the second 17β-hydroxy oxygen, with the alkyl group preferably having less than 13 carbon atoms, and preferably less than four carbon atoms. For example, the alkoxy group may be selected from methoxy, ethoxy, butoxy, isopropoxy, isobutoxy, t-butoxy, valeroxy, hexanoxy, heptanoxy, octanoxy, nonanoxy, decanoxy, undecanoxy, cyclopentoxy, and cyclopentylpropoxy, and may be straight chain, branched or cyclic. The second prohormone comprises 17β-alkoxy-3β-hydroxy-androst-4-ene. The second prohormone thus may comprise 3,17β-di(alkoxy)-androst-4-ene, 17β-alkoxy-3β-hydroxy-estr-4-ene, and 3,17β-di(alkoxy)-estr-4-ene.

Alkoxy groups may be prepared as described above.

According to another embodiment of this aspect of the invention, the second 17-position promoiety and the second 17β-hydroxy oxygen appended to the second 17 position establish an alkoxymethyloxy group of an alkoxy methyl ether, with the alkoxy moiety having an alkyl moiety having less than 13 carbon atoms. More preferably, the alkoxy moiety consists of methoxy. Other representative alkoxy moieties suitable for the invention include moieties selected from the group consisting of ethoxy, butoxy, isopropoxy, isobutoxy, t-butoxy, valeroxy, hexanoxy, heptanoxy, octanoxy, nonanoxy, decanoxy, undecanoxy, cyclopentoxy, and cyclopentylpropoxy. Examples of second prohormones therefore include, for example, 17β-alkoxymethyl-3β-hydroxy-androst-4-ene (e.g., 17β-methoxymethyl-5α-androst-4-ene-3-ol), 17β-alkoxymethyl-3β-hydroxy-estr-4-ene (e.g., 17β-methoxymethyl-5α-estr-4-ene-3-ol), 3,17β-di(alkoxymethyl)-5α-androst-4-ene (e.g., 3,17β-di(methoxymethyl)-5α-androst-4-ene), 3,17β-di(alkoxymethyl)-5α-estr-4-ene (e.g., 3,17β-di(methoxymethyl)-5α-estr-4-ene), and mixtures or combinations thereof. In each instance in which the second prohormone includes a hydroxy group at the 3 position, the bond at the 3 carbon may be 3α or 3β and preferably comprises a mixture thereof.

Methoxymethyl ethers of these types may be synthesized using known synthesis techniques, for example, by reaction with iodomethyl methyl ether, or chloromethyl methyl ether, in a Williamson ether synthesis. The prohormones may be formed by reaction of the sodium or potassium salt of the parent androgen with chloromethyl methyl ether, bromomethyl methyl ether, or iodomethyl methyl ether in dichloromethane or other suitable solvent.

Preferred combinations of first and second prohormones in the composition of this aspect of the invention include first and second prohormones that each have alkyloxycarbonyloxy groups at the 17β position. The first prohormone preferably is selected from the group consisting of 5α-androst-1-ene-3-ol-17β-alkylcarbonate, 5α-androst-1-ene-3-one-17β-alkylcarbonate, 5α-androst-1-ene-3,17β-di(alkylcarbonate), 5α-estr-1-ene-3-ol-17β-alkylcarbonate, 5α-estr-1-ene-3-one-17β-alkylcarbonate, and 5α-estr-1-ene-3,17β-di(alkylcarbonate). The second prohormone preferably is selected from the group consisting of androst-4-ene-3-ol-17 β-alkylcarbonate, androst-4-ene-3,17β-di(alkylcarbonate), estr-4-ene-3-ol-17-alkylcarbonate, and estr-4-ene-3,17β-di(alkylcarbonate). The alkyl moiety of the alkyloxycarbonyloxy group is preferably ethyl or propyl.

The composition may be contained or encapsulated by an enteric coating. The composition also may be administered with a carrier, which may comprise a solid carrier, a semi-solid carrier, or a liquid carrier. A preferred liquid carrier is an aqueous emulsion including fatty acid ethyl esters, polysorbate 60, lecithin, and cholesterol or an oil. Another preferred liquid carrier comprises water, glycerin, polysorbate, lecithin, sodium benzoate, ethylene diamine tetraacetic acid (“EDTA”), potassium sorbate, grapefruit seed extract, and vegetable gum.

The composition preferably but optionally may be used for treatment of a human being to supplement or increase the concentration of the parent androgen in vivo. This is not necessarily limiting, however, and veterinary applications, for example, also are possible in certain instances.

In accordance with a second aspect of the invention, a method is provided for administering the composition of the first aspect of the invention into a subject, preferably a human being. The first and second prohormones selected for this method may be any combination of the prohormones described above in connection with the first aspect of the invention.

In an especially preferred embodiment of the second aspect of the invention, the method comprises converting the first prohormone and the second prohormone to their respective parent androgens in a subject in vivo. The subject preferably but optionally is a human being, and the in vivo conversion thus correspondingly comprises converting the prohormone into the parent androgen in vivo within the human being. The method according to this preferred embodiment of the second aspect of the invention comprises administering to the subject a composition comprising a first prohormone and a second prohormone, or a plurality of these, and converting the prohormones to their respective parent androgens in vivo. The first prohormone and the second prohormone may as described above, in any combination.

In accordance with a third aspect of the invention, a composition is provided, wherein the composition comprises:

(a) a first compound having a first skeletal structure as follows

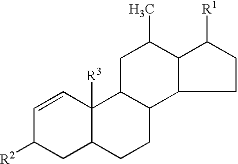

wherein R¹ and R² are the same or different from each other and selected from the group consisting of —OH, —OC(O)OR⁴, —OC(O)R⁴, —OR⁴, —OCH₃OR⁴, and ═O

wherein R³ is selected from the group consisting of hydrogen and methyl, and

wherein R⁴ is an alkyl group having less than 13 carbon atoms; and

(b) a second compound having a second skeletal structure as follows

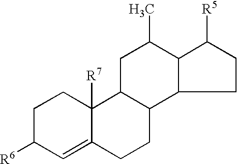

wherein R⁵ and R⁶ are the same or different from each other and selected from the group consisting of —OH, —OC(O)OR⁸, —OC(O)R⁸, —OR⁸, and —OCH₃OR⁸, except that at least one of R⁵ and R⁶ is a member other than —OH,

wherein R⁷ is selected from the group consisting of hydrogen and methyl, and

wherein R⁸ is an alkyl group having less than 13 carbon atoms.

According to one embodiment of this third aspect, a member selected from the group consisting of R¹ and R² consists of ═O. In this embodiment, it is preferred that R² consists of ═O, and R¹ consists of a member selected from the group consisting of hydroxyl, —OC(O)OR⁴ (alkyloxycarbonyloxy) —OC(O)R⁴ (alkanoyloxy), —OR⁴ (alkoxy), and —OCH₃OR⁴ (alkoxymethyloxy).

In another embodiment of this third aspect, the first prohormone is not a prodrug. For example, both R¹ and R² may be —OH. Examples include 5α-androst-1-ene-3,17-diol and 5α-estr-1-ene-3,17-diol, as well as 5α-androst-1-ene-3,17-dione and 5α-estr-1-ene-3,17-dione.

In an especially referred embodiment of this aspect of the invention, R¹ and R² each consists of —OC(O)OR⁴. This preferred embodiment is preferably characterized by R⁴ consisting of a straight-chain alkyl group.

In another preferred embodiment of this third aspect of the invention, R¹ consists of —OC(O)OR⁴ and R² consists of the hydroxyl. It is especially preferred in this embodiment for R⁴ to consist of a straight-chain alkyl group.

Preferably, but not necessarily, R² is —OH, ═O, or the same as R¹.

According to another embodiment of this third aspect, a member selected from the group consisting of R⁵ and R⁶ consists of —OH. In this embodiment, it is preferred that R² consists of —OH, and R¹ consists of a member selected from the group consisting of hydroxyl, —OC(O)OR⁴, —OC(O)R⁴, —OR⁴, and —OCH₃OR⁴, in which R⁴ is a straight chain alkyl.

In an especially referred embodiment of this aspect of the invention, R⁵ and R⁶ each consists of —OC(O)OR⁸. This preferred embodiment is preferably characterized by R⁸ consisting of a straight-chain alkyl group.

Preferably, but not necessarily, R⁶ is —OH, or the same as R⁵.

In one preferred embodiment of the third aspect of the invention, R¹ of the first promoiety is —OC(O)OR⁴, R² is selected from —OH and —OC(O)OR⁴, R⁵—OC(O)OR⁸, and R⁶ is selected from —OH and —OC(O)OR⁸. In these preferred combinations, R⁴ and R⁸ are the same or different, and preferably are selected from —CH₂CH₃ and —CH₂CH₂CH₃.

The composition according to this third aspect of the invention also may comprise a carrier, such as those forms noted above.

In accordance with a fourth aspect of the invention, a method is provided for administering the composition of the third aspect of the invention into a subject, preferably a human being. The first and second prohormones selected for this method may be any combination of the prohormones described above in connection with the third aspect of the invention.

In each of the aforementioned methods, the composition administration may comprise peroral administration, pernasal administration, transdermal administration, sublingual administration, and other means. The administration of the composition also may be by combinations of these techniques or approaches. Peroral administration is presently preferred.

As part of the method, the composition administration may comprise complexing the composition with an hydroxypropyl beta cyclodextrin, and/or with an hydroxypropyl gamma cyclodextrin. This is particularly applicable if administered sublingually. The composition administration optionally may further include applying an enteric coating to the composition prior to administering the composition.

When administered orally or sublingually, the composition enters the gastrointestinal (“GI”) tract, and ultimately the blood stream. Through more direct methods such as through pernasal, transdermal or intravenous injection, the composition enters directly into the blood stream. In each of the instances, the composition may react to form the parent androgen or a prodrug of the parent androgen.

One limitation of known prodrugs of steroids is that, once they are transformed into the parent steroid, they are broken down in the body, and particularly in the liver. This breakdown reduces in vivo concentration and bioavailability of the steroid. In the presently preferred embodiments of the invention, the Class I and Class II compounds are derived from prohormones less prone to such breakdown in the body relative to many known hormones and prodrug-type compounds. This in many instances is attributable to the promoiety, which makes the compound more resistant to hydrolysis and other reactions that inhibit or destroy them prematurely in the body. In vivo concentrations thus can be maintained more readily, and bioavailability of the parent androgen can be improved.

The composition preferably is administered in amounts effective to supplement or increase the concentration of the parent androgen in vivo. The appropriate dosage therefore may take into account natural or otherwise expected variations in androgen concentration, such as the normal daily variations in natural androgen production and consumption, and such as normal variations in in vivo androgen concentrations over days or weeks.

According to a preferred embodiment of the method, the composition may be administered using a dosage given periodically for, for example, a maximum of two weeks, followed by a period, for example, of at least two weeks, of non-administration to permit recovery of natural parent androgen production in the subject, for example, to levels comparable to the baseline level possessed by the subject prior to use of the composition. This schedule can permit the composition to supplement or increase the concentration of the parent androgen in vivo for an effective period, and then terminate further dosages of the composition as its effectiveness attenuates.

The preferred dosage of the compound will depend upon the specific first and second prohormones, the subject or class of subject to which the composition is to be administered, and other factors commonly affecting dosage determinations for this type of composition. In general, the dosage should be such that a sufficient amount of the composition enters the system of the subject and supplements or increases the natural it vivo concentration of the parent androgen.

In accordance with presently preferred versions of the inventive compositions and methods, as described above for the various aspects of the invention, the composition administration, particularly when applied to humans, comprises administering the first and second prohormones in a total amount ranging from 1.0 mg to 1 gram mg per day, more preferably in an amount ranging from 50 mg to 600 mg per day, and even more preferably in an amount ranging from 300 mg to 600 mg per day.

The mass ratio of first prohormone to second prohormone in the various compositions and methods of this invention is preferably, but not necessary, 1:5 to 3:1, more preferably 1:2 to 2:1.

The compound administration also may comprise administering the compound only in morning-time.

Advantages of the preferred compounds and methods according to the invention flow from the advantageous or desirable effects of the parent androgen, as well as from the increased in vivo concentration and bioavailability of the prodrug and/or the corresponding parent androgen resulting from administration of the composition. The parent androgens can provide pro-athletic and anabolic effects. In some instances, the parent androgens can provide improved immune system efficacy and/or mood enhancement as well.

Carriers as described above for the other aspects of the invention apply to the composition according to the methods described herein as well.

The following examples are illustrative, and are not exhaustive or necessarily limiting as to the scope of the present invention.

### EXAMPLE 1

To 10 ml dichloromethane was added 1.0 gram androst-4-ene-3,17-diol and 2.00 equivalents each of ethyl chloroformate and 2,6-lutidine. This was then stirred overnight, diluted with an additional 60 ml of dichloromethane, and filtered. The filtrate was washed twice in a separatory funnel with acidic water, once with neutral water, then dried over sodium sulfate and placed in a freezer at −10 C for two hours. The resulting white colorless crystals were recovered by filtration, yielding 0.47 grams of androst-4-ene-3,17β-di(ethylcarbonate) ester.

Pyridine and/or triethylamine may be used as alternate bases. Alternate solvents include acetone, acetonitrile, pyridine, and lutidine. Pyridine is especially preferred.

### EXAMPLE 2

About 100 mg of a second prohormone of androst-4-ene-3,17β-di(ethylcarbonate) and about 200 mg of a first prohormone of 5α-androst-1-ene-3-one-17β-ethylcarbonate ester were added to a liquid carrier component comprising 2,876 mg of purified water, 2,443 mg of glycerin, 1,847 mg of proplyene glycol, 459 mg of polysorbate, 396 mg of Lecithin, 202 mg of EDTA, 185 mg of vegetable gum, 566 mg of natural flavor blend, 96 mg of sodium benzoate, 96 mg of potassium sorbate, and 96 mg of calcium propionate. The components were stirred to achieve dissolution and thus form the resultant solution. This specific example yielded approximately 9,620 mg or about 9 ml of the solution. This solution can be taken by a human subject in daily doses of about 9-18 ml.

### EXAMPLE 3

About 150 mg of androst-4-ene-3,17β-di(ethylcarbonate) and about 150 mg of 5α-androst-1-ene-3-one-17β-ethylcarbonate ester are added to a liquid carrier component comprising 2,876 mg of purified water, 2,443 mg of glycerin, 1,847 mg of proplyene glycol, 459 mg of polysorbate, 396 mg of Lecithin, 202 mg of EDTA, 185 mg of vegetable gum, 566 mg of natural flavor blend, 96 mg of sodium benzoate, 96 mg of potassium sorbate, and 96 mg of calcium propionate. The components were stirred to achieve dissolution and thus form the resultant solution. This specific example yielded approximately 9,620 mg or about 9 ml of the solution. This solution can be taken by a human subject in daily doses of about 9-18 ml.

### EXAMPLE 4

About 200 mg of androst-4-ene-3,17β-di(ethylcarbonate) and about 100 mg of 5α-androst-1-ene-3-one-17β-ethylcarbonate ester were added to a liquid carrier component comprising 2,876 mg of purified water, 2,443 mg of glycerin, 1,847 mg of proplyene glycol, 459 mg of polysorbate, 396 mg of Lecithin, 202 mg of EDTA, 185 mg of vegetable gum, 566 mg of natural flavor blend, 96 mg of sodium benzoate, 96 mg of potassium sorbate, and 96 mg of calcium propionate. The components were stirred to achieve dissolution and thus form the resultant solution. This specific example yielded approximately 9,620 mg or about 9 ml of the solution. This solution can be taken by a human subject in daily doses of about 9-18 ml.

### EXAMPLE 5

About 150 mg of androst-4-ene-3,17β-di(ethylcarbonate) and about 150 mg of 5α-androst-1-ene-3-one-17β ethylcarbonate ester were added to a liquid carrier component comprising 2,150 mg of purified water, 2,800 mg of glycerin, 2,216 mg of proplyene glycol, 459 mg of polysorbate, 396 mg of Lecithin, 202 mg of EDTA, 185 mg of vegetable gum, 566 mg of natural flavor blend, 96 mg of sodium benzoate, 96 mg of potassium sorbate, and 96 mg of calcium propionate. The components were stirred to achieve dissolution. This yielded approximately 9,620 mg or about 9 ml of the solution. This solution can be taken by a human subject in daily doses of about 9-18 ml.

### EXAMPLE 6

About 150 mg of androst-4-ene-3,17β-diol 3,17β-di(ethylcarbonate) and about 150 mg of 17β-hydroxyandrost-1-ene-3-one 17β-ethylcarbonate ester were added to a liquid carrier component comprising 3,550 mg of purified water, 2,132 mg of glycerin, 1,484 mg of proplyene glycol, 459 mg of polysorbate, 396 mg of Lecithin, 202 mg of EDTA, 185 mg of vegetable gum, 566 mg of natural flavor blend, 96 mg of sodium benzoate, 96 mg of potassium sorbate, and 96 mg of calcium propionate. The components were stirred to achieve dissolution. This yielded approximately 9,620 mg or about 9 ml of the solution. This solution can be taken by a human subject in daily doses of about 9-18 ml.

### EXAMPLE 7

About 100 mg of a first component of estr-4-ene-3,17β-diol-3,17β di(ethylcarbonate) and about 200 mg of a second component of 17β-hydroxyandrost-1-ene-3-one 17β-ethylcarbonate ester were added to a liquid carrier component comprising 2,876 mg of purified water, 2,443 mg of glycerin, 1,847 mg of proplyene glycol, 459 mg of polysorbate, 396 mg of Lecithin, 202 mg of EDTA, 185 mg of vegetable gum, 566 mg of natural flavor blend, 96 mg of sodium benzoate, 96 mg of potassium sorbate, and 96 mg of calcium propionate. The components were stirred to achieve dissolution and thus form the resultant solution. This specific example yielded approximately 9,620 mg or about 9 ml of the solution. This solution can be taken by a human subject in daily doses of about 9-18 ml.

### EXAMPLE 8

About 150 mg of estr-4-ene-3,17β-diol-3,17β di(ethylcarbonate) and about 150 mg of 17β-hydroxyandrost-1-ene-3-one-17β-ethylcarbonate ester {?} were added to a liquid carrier component comprising 2,876 mg of purified water, 2,443 mg of glycerin, 1,847 mg of proplyene glycol, 459 mg of polysorbate, 396 mg of Lecithin, 202 mg of EDTA, 185 mg of vegetable gum, 566 mg of natural flavor blend, 96 mg of sodium benzoate, 96 mg of potassium sorbate, and 96 mg of calcium propionate. The components were stirred to achieve dissolution and thus form the resultant solution. This yielded approximately 9,620 mg or about 9 ml of the solution, which can be taken by a human subject in daily doses of about 9-18 ml.

### EXAMPLE 9

About 200 mg of estr-4-ene-3,17β-diol-3,17β-di(ethylcarbonate) and about 100 mg of 17β-hydroxyandrost-1-ene-3-one-17β-ethylcarbonate ester were added to a liquid carrier component comprising 2,876 mg of purified water, 2,443 mg of glycerin, 1,847 mg of proplyene glycol, 459 mg of polysorbate, 396 mg of Lecithin, 202 mg of EDTA, 185 mg of vegetable gum, 566 mg of natural flavor blend, 96 mg of sodium benzoate, 96 mg of potassium sorbate, and 96 mg of calcium propionate. The components were stirred to achieve dissolution and thus form the resultant solution. This specific example yielded approximately 9,620 mg or about 9 ml of the solution. This can be taken by a human subject in daily doses of about 9-18 ml.

### EXAMPLE 10

About 150 mg of estr-4-ene-3,17β-diol-3,17β-di(ethylcarbonate) and about 150 mg of 17β-hydroxyandrost-1-ene-3-one-17β-ethylcarbonate ester were added to a liquid carrier component comprising 2,150 mg of purified water, 2,800 mg of glycerin, 2,216 mg of proplyene glycol, 459 mg of polysorbate, 396 mg of Lecithin, 202 mg of EDTA, 185 mg of vegetable gum, 566 mg of natural flavor blend, 96 mg of sodium benzoate, 96 mg of potassium sorbate, and 96 mg of calcium propionate. The components were stirred to achieve dissolution. This yielded approximately 9,620 mg or about 9 ml of the solution. This solution can be taken by a human subject in daily doses of about 9-18 ml.

### EXAMPLE 11

About 150 mg of estr-4-ene-3,17β-diol-3,17β-di(ethylcarbonate) and about 150 mg of 17β-hydroxyandrost-1-ene-3-one-17β-ethylcarbonate ester were added to a liquid carrier component comprising 3,550 mg of purified water, 2,132 mg of glycerin, 1,484 mg of proplyene glycol, 459 mg of polysorbate, 396 mg of Lecithin, 202 mg of EDTA, 185 mg of vegetable gum, 566 mg of natural flavor blend, 96 mg of sodium benzoate, 96 mg of potassium sorbate, and 96 mg of calcium propionate. The components were stirred to achieve dissolution. This yielded approximately 9,620 mg or about 9 ml of the solution. This solution can be taken by a human subject in daily doses of about 9-18 ml.

Additional advantages and modifications will readily occur to those skilled in the art. Therefore, the invention in its broader aspects is not limited to the specific details, representative devices and methods, and illustrative examples shown and described. Accordingly, departures may be made from such details without departing from the spirit or scope of the general inventive concept as defined by the appended claims and their equivalents.

What is claimed is:

1. A composition comprising:
(a) a first prohormone for increasing the concentration of a Class I parent androgen in a subject in vivo, the Class I parent androgen comprising a member selected from the group consisting of 5α-androst-1-ene-3α,17β-diol, 5α-androst-1-ene-3β,17β-diol, 5α-estr-1-ene-3α,17β-diol, 5α-estr-1-ene-3β,17β-diol, 17β-hydroxy-5α-androst-1-ene-3-one, and 17β-hydroxy-5α-estr-1-ene-3-one, the Class I parent androgen having a first skeletal structure comprising a 1 position and a 17 position and the Class I parent androgen further comprising a 17β-hydroxy group comprising a 17β-hydroxy oxygen appended to the 17 position and a 17β-hydroxy hydrogen appended to the 17β-hydroxy oxygen, the first prohormone comprising
(i) a first substrate having the first skeletal structure of the Class I parent androgen comprising a 1 position and a first 17 position corresponding to the 1 and 17 positions respectively of the Class I parent androgen first skeletal structure, the first substrate comprising a first carbon-carbon double bond at the 1 position and a first 17β-hydroxy oxygen appended to the first 17 position; and
(ii) a first 17-position promoiety appended to the first 17β-hydroxy oxygen of the substrate as a substitute for the 17β-hydroxy hydrogen of the Class I parent androgen; and
(b) a second prohormone for increasing the concentration of a Class II parent androgen in the subject in vivo, the Class II parent androgen comprising a member selected from the group consisting of androst-4-ene-3α,17β-diol, androst-4-ene-3β,17β-diol, estr-4-ene-3α,17β-diol, and estr-4-ene-3β,17β-diol, the Class II parent androgen having a second skeletal structure comprising a 4 position and a 17 position and the Class II parent androgen further comprising a 17β-hydroxy group comprising a 17β-hydroxy oxygen appended to the 17 position and a 17β-hydroxy hydrogen appended to the 17β-hydroxy oxygen, the second prohormone comprising
(i) a second substrate having the second skeletal structure of the Class II parent androgen comprising a 4 position and a second 17 position corresponding to the 4 and 17 positions respectively of the Class II parent androgen second skeletal structure, the second substrate comprising a second carbon-carbon double bond at the 4 position and a second 17β-hydroxy oxygen appended to the second 17 position; and
(ii) a second 17-position promoiety appended to the second 17β-hydroxy oxygen of the substrate as a substitute for the 17β-hydroxy hydrogen of the Class II parent androgen.

2. A composition according to claim 1, wherein:
the Class I parent androgen comprises a member selected from the group consisting of 5α-androst-1-ene-3α,17β-diol, 5α-androst-1-ene-3β,17β-diol, 5α-estr-1-ene-3α,17β-diol, and 5α-estr-1-ene-3β,17β-diol;
the first skeletal structure of the Class I parent androgen further comprises a 3 position, and the Class I parent androgen further comprises a 3-hydroxy group comprising a 3-hydroxy oxygen appended to the 3 position and a 3-hydroxy hydrogen appended to the 3-hydroxy oxygen;
the first substrate comprises a 3 position corresponding to the 3 position of the Class I parent androgen skeletal structure, the first substrate comprising a 3-hydroxy oxygen appended to the 3 position; and
the first prohormone further comprises a 3-position promoiety appended to the 3β-hydroxy oxygen of the first substrate as a substitute for the 3-hydroxy hydrogen of the Class I parent androgen.

3. A composition according to claim 1, wherein:
the second skeletal structure of the Class II parent androgen further comprises a 3 position, and the Class II parent androgen further comprises a 3-hydroxy group comprising a 3-hydroxy oxygen appended to the 3 position and a 3-hydroxy hydrogen appended to the 3-hydroxy oxygen;
the second substrate comprises a 3 position corresponding to the 3 position of the Class II parent androgen second skeletal structure, the second substrate comprising a 3-hydroxy oxygen appended to the 3 position; and
the second prohormone further comprises a 3-position promoiety appended to the 3β-hydroxy oxygen of the second substrate as a substitute for the 3-hydroxy hydrogen of the Class II parent androgen.

4. A composition according to claim 1, wherein:
the Class I parent androgen comprises a member selected from the group consisting of 5α-androst-1-ene-3α,17β-diol, 5α-androst-1-ene-3β,17β-diol, 5α-estr-1-ene-3α,17β-diol, and 5α-estr-1-ene-3β,17β-diol;
the first skeletal structure of the Class I parent androgen further comprises a 3 position, and the Class I parent androgen further comprises a 3-hydroxy group comprising a 3-hydroxy oxygen appended to the 3 position and a 3-hydroxy hydrogen appended to the 3-hydroxy oxygen;
the first substrate comprises a first 3 position corresponding to the 3 position of the Class I parent androgen skeletal structure, the first substrate comprising a first 3-hydroxy oxygen appended to the first 3 position;
the first prohormone further comprises a first 3-position promoiety appended to the first 3β-hydroxy oxygen of the substrate as a substitute for the 3-hydroxy hydrogen of the Class I parent androgen;
the second skeletal structure of the Class II parent androgen further comprises a 3 position, and the Class II parent androgen further comprises a 3-hydroxy group comprising a 3-hydroxy oxygen appended to the 3 position and a 3-hydroxy hydrogen appended to the 3-hydroxy oxygen;
the second substrate comprises a second 3 position corresponding to the second 3 position of the Class II parent androgen skeletal structure, the second substrate comprising a second 3-hydroxy oxygen appended to the second 3 position; and
the second prohormone further comprises a 3-position promoiety appended to the second 3β-hydroxy oxygen of the second substrate as a substitute for the 3-hydroxy hydrogen of the Class II parent androgen.

5. A composition according to claim 1, wherein the first 17-position promoiety and the first 17β-hydroxy oxygen appended to the first 17 position establish an alkyloxycarbonyloxy group of an alkylcarbonate ester.

6. A composition according to claim 5, wherein the first prohormone comprises 5α-estr-1-ene-3,17β-di(alkylcarbonate).

7. A composition according to claim 5, wherein the first prohormone comprises 5α-estr-1-ene-3,17β-di(ethylcarbonate).

8. A composition according to claim 1, wherein the first 17-position promoiety and the first 17β-hydroxy oxygen appended to the first 17 position establish an alkanoyloxy group.

9. A composition according to claim 1, wherein the first 17-position promoiety and the first 17β-hydroxy oxygen appended to the first 17 position establish an alkoxy group.

10. A composition according to claim 1, wherein the first 17-position promoiety and the first 17β-hydroxy oxygen appended to the first 17 position establish an alkoxymethyloxy group.

11. A composition according to claim 1, wherein the second 17-position promoiety and the second 17β-hydroxy oxygen appended to the second 17 position establish an alkyloxycarbonyloxy group.

12. A composition according to claim 1, wherein the second 17-position promoiety and the second 17β-hydroxy oxygen appended to the second 17 position establish an alkanoyloxy group.

13. A composition according to claim 1, wherein the second 17-position promoiety and the second 17β-hydroxy oxygen appended to the second 17 position establish an alkoxy group.

14. A composition according to claim 1, wherein the second 17-position promoiety and the second 17β-hydroxy oxygen appended to the second 17 position establish an alkoxymethyloxy group.

15. A composition according to claim 1, wherein:
the first prohormone comprises a member selected from the group consisting of 5α-androst-1-ene-3-one-17β-alkylcarbonate, 5α-androst-1-ene-3-hydroxy-17β-alkylcarbonate, and 5α-androst-1-ene-3,17β-di(alkylcarbonate); and
the second prohormone comprises a member selected from the group consisting of androst-4-ene-3β-hydroxy-17β-alkylcarbonate and androst-4-ene-3,17β-di(alkylcarbonate).

16. A composition according to claim 1, wherein the first prohormone comprises 5α-androst-1-ene-3-one-17β-ethylcarbonate and the second member comprises a member selected from the group consisting of androst-4-ene-3-hydroxy-17β-ethylcarbonate and androst-4-ene-3,17β-di(ethylcarbonate).

17. A composition comprising:
(a) a first compound having a first skeletal structure as follows 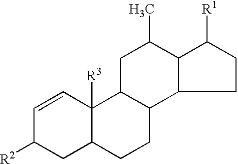
wherein R¹ and R² are the same or different from one another and selected from the group consisting of hydroxyl, —OC(O)OR⁴, —OC(O)R⁴, —OR⁴, —OCH₂OR⁴, and ═O,
wherein R³ is selected from the group consisting of hydrogen and methyl, and wherein R⁴ is an alkyl group having less than 13 carbon atoms; and
(b) a second compound having a second skeletal structure as follows 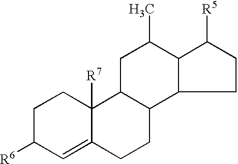
wherein R⁵ and R⁶ are the same or different from one another and selected from the group consisting of hydroxyl, —OC(O)OR⁸, —OC(O)R⁸, —OR⁸, and —OCH₂OR⁸, except that R⁵ and R⁶ are not both hydroxyl,
wherein R⁷ is selected from the group consisting of hydrogen and methyl, and
wherein R⁸ is an alkyl group having less than 13 carbon atoms.

18. A composition according to claim 17, wherein a member selected from the group consisting of R¹ and R² consists of ═O.

19. A composition according to claim 17, wherein R² consists of ═O, and wherein R¹ consists of a member selected from the group consisting of hydroxyl, —OC(O)OR⁴, —OC(O)R⁴, —OR⁴, and —OCH₂OR⁴.

20. A composition according to claim 17, wherein R¹ and R² each consists of —OC(O)OR⁴.

21. A composition according to claim 20, wherein R⁴ consists of a straight-chain alkyl group.

22. A composition according to claim 17, wherein R¹ consists of —OC(O)OR⁴ and R² consists of the hydroxyl.

23. A composition according to claim 22, wherein R⁴ consists of a straight-chain alkyl group.

24. A composition according to claim 17, wherein a member selected from the group consisting of R¹ and R² consists of the hydroxyl.

25. A composition according to claim 17, wherein R⁵ and R⁶ each consists of —OC(O)OR⁸.

26. A composition according to claim 25, wherein R⁸ consists of a straight-chain alkyl group.

27. A composition according to claim 17, wherein R⁵ consists of —OC(O)OR⁸ and R⁶ consists of the hydroxyl.

28. A composition according to claim 27, wherein R⁸ consists of a straight-chain alkyl group.

29. A method for increasing androgen concentration of a subject, said method comprising:
(a) providing a composition comprising
(i) a first prohormone for increasing the concentration of a Class I parent androgen in a subject in vivo, the Class I parent androgen comprising a member selected from the group consisting of 5α-androst-1-ene-3α,17β-diol, 5α-androst-1-ene-3β,17β-diol, 5α-estr-1-ene-3α,17β-diol, 5α-estr-1-ene-3β,17β-diol, and 17β-hydroxy-5α-androst-1-ene-3-one, the Class I parent androgen having a first skeletal structure comprising a 1 position and a 17 position and the Class I parent androgen further comprising a 17β-hydroxy group comprising a 17β-hydroxy oxygen appended to the 17 position and a 17β-hydroxy hydrogen appended to the 17β-hydroxy oxygen, the first prohormone comprising
(A) a first substrate having the first skeletal structure of the Class I parent androgen comprising a 1 position and a first 17 position corresponding to the 1 and 17 positions respectively of the Class I parent androgen first skeletal structure, the first substrate comprising a first carbon-carbon double bond at the 1 position and a first 17β-hydroxy oxygen appended to the first 17 position; and
(B) a first 17-position promoiety appended to the first 17β-hydroxy oxygen of the substrate as a substitute for the 17β-hydroxy hydrogen of the Class I parent androgen; and
(ii) a second prohormone for increasing the concentration of a Class II parent androgen in a subject in vivo, the Class II parent androgen comprising a member selected from the group consisting of androst-4-ene-3α,17β-diol, androst-4-ene-3β,17β-diol, estr-4-ene-3α,17β-diol, and estr-4-ene-3β,17β-diol, the Class II parent androgen having a second skeletal structure including a 4 position and a 17 position and the Class II parent androgen further comprising a 17β-hydroxy group comprising a 17β-hydroxy oxygen appended to the 17 position and a 17β-hydroxy hydrogen appended to the 17β-hydroxy oxygen, the second prohormone comprising
(A) a second substrate having the second skeletal structure of the Class II parent androgen comprising a 4 position and a second 17 position corresponding to the 4 and 17 positions respectively of the Class II parent androgen second skeletal structure, the second substrate comprising a second carbon-carbon double bond at the 4 position and a second 17β-hydroxy oxygen appended to the second 17 position; and
(B) a second 17-position promoiety appended to the second 17β-hydroxy oxygen of the substrate as a substitute for the 17β-hydroxy hydrogen of the Class II parent androgen; and
(b) administering the composition to the subject.

30. A method for increasing androgen concentration of a human being, said method composition comprising:
(a) providing a composition comprising
(i) a first compound having a first skeletal structure as follows 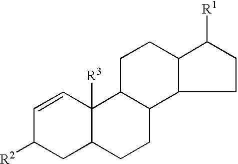
wherein R¹ and R² are the same or different from one another and selected from the group consisting of —OH, —OC(O)OR⁴, —OC(O)R⁴, —OR⁴, —OCH₂OR⁴, and ═O,
wherein R³ is selected from the group consisting of hydrogen and methyl, and
wherein R⁴ is an alkyl group having less than 13 carbon atoms; and
(ii) a second compound having a second skeletal structure as follows 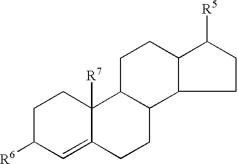
wherein R⁵ and R⁶ are the same or different from one another and selected from the group consisting of —OH, —OC(O)OR⁸, —OC(O)R⁸, —OR⁸, and —OCH₂OR⁸, except that at least one of R⁵ and R⁶ is other than —OH, wherein R⁷ is selected from the group consisting of hydrogen and methyl, and
wherein R⁸ is an alkyl group having less than 13 carbon atoms; and
(b) administering the composition to a human.

31. A composition comprising:
(a) a prohormone for increasing the concentration of a Class I parent androgen in a subject in vivo, the Class I parent androgen comprising a member selected from the group consisting of 5α-androst-1-ene-3α,17β-diol, 5α-androst-1-ene-3β,17β-diol, 5α-estr-1-ene-3α,17β-diol, 5α-estr-1-ene-3β,17β-diol, 17β-hydroxy- 5α-androst-1-ene-3-one, and 17β-hydroxy-5α-estr-1-ene-3-one, the Class I parent androgen having a skeletal structure comprising a 1 position and a 17 position and the Class I parent androgen further comprising a 17β-hydroxy group comprising a 17β-hydroxy oxygen appended to the 17 position and a 17β-hydroxy hydrogen appended to the 17β-hydroxy oxygen, the prohormone comprising
(i) a substrate having the skeletal structure of the Class I parent androgen comprising a 1 position and a 17 position corresponding to the 1 and 17 positions respectively of the Class I parent androgen skeletal structure, the substrate comprising a carbon-carbon double bond at the 1 position and a 17β-hydroxy oxygen appended to the 17 position, and
(ii) a 17-position promoiety appended to the 17β-hydroxy oxygen of the substrate as a substitute for the 17β-hydroxy hydrogen of the Class I parent androgen; and
(b) pregnenolone.
